(19) United States
(12) Patent Application Publication
(10) Pub. No.: US 2025/0342975 A1
(43) Pub. Date: Nov. 6, 2025

(54) METHOD AND SYSTEM FOR AUTOMATICALLY ASSISTING MEDICAL PRACTITIONER

(71) Applicant: INNOVACCER INC., San Francicso, CA (US)

(72) Inventors: Kuldeep SINGH, San Francisco, CA (US); Ankit MAHESHWARI, Noida (IN); Rajat GOYAL, Noida (IN); Nilanjana BHATTACHARYYA, Noida (IN)

(21) Appl. No.: 18/763,800

(22) Filed: Jul. 3, 2024

(30) Foreign Application Priority Data
May 2, 2024 (IN) 202411035004

(51) Int. Cl. G16H 80/00; A61B 5/00; G06F 3/01; G06F 40/103; G06F 40/166; G10L 17/02; G10L 17/22; G16H 10/60

(52) U.S. Cl. CPC G16H 80/00; A61B 5/4803; G06F 3/017; G06F 40/103; G06F 40/166; G10L 17/02; G10L 17/22; G16H 10/60

(57) ABSTRACT

A system and a method for automatically assisting physicians during patient encounters includes transcribing and diarizing physician-patient interactions, extracting clinical concepts, and combining data with patient history data to provide suggestions to the physicians. A speech and gesture recognition module captures audio from patient-doctor interactions and transcribes the audio into text in real-time. A natural language processing module diarizes the transcribed text to attribute speech. A clinical concept extraction module identifies and extracts clinical concepts from transcription. A clinical recommendation module integrates real-time data with historical patient records and analyzes the combined data set to generate evidence-based suggestions to the physician. A physician's own historical data is validated to generate a unique profile for each physician. Interactions with the physician are analyzed to understand decision-making patterns. The physician's profile is updated and future physician decisions are forecast based on past behavior.

### CROSS REFERENCE TO RELATED APPLICATION

This application claims benefit of India Application Serial No. 202411035004 filed May 2, 2024, the entire disclosure of which is incorporated herein by reference.

### TECHNICAL FIELD

The present disclosure relates to a method and a system for automatically assisting physicians during patient visit, and more particularly to automatically transcribe and diarize the physician-patient interaction in order to provide decision support to the physician.

### BACKGROUND

The field of medicine has long sought to improve the accuracy and efficiency of patient care. Patients tend to forget as much of the information conveyed by the doctors as soon as they leave the clinic. Most doctors dictate the diagnosis during treatment of the patients for recording the dictations and making them available for patients, as one solution to this problem. In some cases, an interactive voice response to guide the patient in medication and to help the doctor maintain the record is used in practice. Recorded treatment sessions are provided to the patients with added security using a QR code. The dictation of the doctor could be converted to text and a model may interact with users and ask necessary questions to give a comprehensive result using machine learning.

A virtual assistant, also known as a virtual agent, is built into many mobile communications devices, like smartphones. It is designed to accept speech input from a user and use a variety of locally or remotely accessible resources to recognize the user's speech, try to understand the user's intent, and respond by carrying out one or more desired tasks based on that understanding, e.g., perform an internet search, make a phone call, schedule an appointment, etc. Some physicians also have tried to use a recorded conversation model along with a virtual scribe to create Subjective, Objective, Assessment and Plan (SOAP) notes, which can be copied to the Electronic Medical Record (EMR) or in some cases integrated into the Electronic Health Record (EHR) software. Since it involves a scribe to edit the documents, it has all the challenges of involving a human in the process. This also has challenges of integration, which creates its own additional issues.

To support patients, their caregivers, and medical professionals, artificial intelligence (AI) technologies, particularly that use machine learning techniques, are being more and more integrated into many healthcare domains. The main motivations behind employing AI technologies include supporting better decision-making and improving care quality. The existing systems only enable recording of the patient-doctor interaction but does not provide decision support to streamline the documentation process and reduce burnout. One of the challenges in conventional clinical practice is the management of vast amounts of patient data and the need for timely and accurate documentation.

Thus, a heretofore unaddressed need exists in the industry to address the aforementioned deficiencies and inadequacies.

### SUMMARY

In one aspect of the present disclosure, a computer-implemented system for automatically assisting physicians during patient encounters by listening to patient-doctor conversations is configured to transcribe and diarize the interactions, extract clinical concepts, and combine the data with patient history data to provide suggestions to assist physicians. The system comprises a computerized device having at least one non-transitory memory and at least one processor capable of executing instructions stored in the memory. At least one input device is in communication with the memory. The input device is configured to capture at least one of audio data or image data. A compute module stored on the memory is configured to process, interpret, and analyze data to provide accurate and timely assistance to physicians. The compute module comprises a speech and gesture recognition module that is configured to capture audio from a patient-physician interaction captured by the input device. The audio data is transcribed into text in real-time with high accuracy. A natural language processing module that is configured to diarize the transcribed text to attribute speech to a correct speaker. The system further comprises a clinical concept extraction module that is configured to identify and extract clinical concepts from the transcribed text. A clinical recommendation module is configured to integrate real-time data with historical patient records into a combined data set and to analyze the combined data set to generate evidence-based suggestions. The evidence-based suggestions are provided to the physician through a user-friendly interface via a display device.

In an embodiment, the compute module further includes a physician model refinement module that is configured to train and validate using historical data of the physician. The physician model refinement module is configured to create a unique profile for each physician, a tracking module to track every interaction between the physician and the AI system on the computerized device and to analyze feedback from the physician to understand their decision-making patterns. The physician model refinement module is further configured to update the unique profile of the physician based on the analyzed feedback, learn continuously from each interaction between the physician and the computerized device, and refine the physician model over time. The physician model refinement module is also configured to forecast future physician decisions based on the unique profile using predictive analytics to improve a relevance of the evidence-based suggestions over time.

In another embodiment, the system further includes a post-visit summary generation module that is configured to compile a comprehensive visit summary of the patient-physician interaction based on the audio data and the image data of the physician.

In yet another embodiment, a data storage module is configured to store data of the patient-physician interaction and interactions between the physician and the computerized device. The data storage module further includes a physician module comprising at least one of physician feedback, notes, medications or cases. The data storage module further includes a clinical knowledge database module comprising a structured collection of information related to clinical medicine and healthcare. The clinical knowledge database further includes at least one of: medical terminology, disease classifications, diagnostic criteria, treatment guidelines, drug information, and clinical pathways. The data storage module further includes a patient database module configured to store and manage patient-related information comprising: at least one of patient demographics, medical history, diagnoses, treatments, medications, or lab results.

In an embodiment, the system further includes an external source of electronic health records (EHR) enabling the clinical recommendation module to incorporate at least a medical history of the patient into the evidence-based suggestions.

In an embodiment, the speech and gesture recognition module is configured to transcribe one or more accents, dialects, and medical terminologies. The speech and gesture recognition module is further configured to recognize hand gestures and convert said hand gestures to a command for accepting and rejecting the evidence-based suggestions.

In an embodiment, the natural language processing module is configured to process complex medical language and colloquial speech.

In another embodiment, the clinical concept extraction module is configured to map extracted clinical concepts to standardized medical ontologies and codes.

In yet another embodiment, the post-visit summary generation module is configured to format the comprehensive visit summary according to a learned documentation style of the physician and allows for physician review and edits before finalizing the comprehensive visit summary and sending it to the EHR.

In another aspect of the present disclosure, a method for automatically assisting physicians during patient encounters is provided. The method includes the step of using at least one input device of a computerized device, the computerized device having at least one non-transitory memory and at least one processor capable of executing instructions stored in the memory. The at least one input device is in communication with the memory and configured to capture at least one of audio data or image data. The method further comprises the step of capturing audio data from patient-physician interactions and transcribing the audio data into transcribed text in real-time. The method includes the steps of diarizing the transcribed text to attribute speech to a correct speaker using a natural language processing module, and identifying and extracting clinical concepts from the transcribed text. In a preferred embodiment, the patient-physician conversation comprises at least one of clinical terms, patient symptoms, diagnostic information, and treatment options discussed during the patient-doctor interaction. The method further comprises the step of integrating real-time data with historical patient records to form a combined data set and analyzing the combined data set to generate evidence-based suggestions. The evidence-based suggestions are presented to the physician through a user-friendly interface via a display device.

In another embodiment, the method further includes the steps of providing feedback from the physician to the computerized device, creating a unique profile for each physician, and tracking every interaction between the physician and the AI system on the computerized device. The method further includes analyzing the feedback from the physician to understand their decision-making patterns. The physician feedback includes rejecting the evidence-based suggestion, accepting the evidence-based suggestion, and accepting the evidence-based suggestion with additional physician inputs. The method further includes the step of updating the unique profile of the physician based on the analyzed feedback using an algorithm module, and learning continuously from each interaction between the physician and the computerized device, allowing it to refine the unique profile in the doctor-specific model over time. The method further includes forecasting a future physician decision based on the unique profile, and improving a relevance of the evidence-based suggestions over time. The method further includes the step of storing data of the patient-physician interaction and interactions between the physician and the computerized device in a data storage module. The method also includes the step of generating a comprehensive visit summary based on the unique profile.

In an embodiment, the data storage module comprises at least one of physician feedback, notes, medications, or cases.

In an embodiment, the data storage module further comprises a clinical knowledge database module that comprises at least one of: medical terminology, disease classifications, diagnostic criteria, treatment guidelines, drug information, or clinical pathways.

Further, another embodiment of the method includes the step of storing and managing patient-related information by a patient database module. The patient-related information comprises at least one of: patient demographics, medical history, diagnoses, treatments, medications, or lab results.

Numerous additional features, embodiments, and benefits of the methods and system of the present disclosure are discussed below in the detailed description which follows.

### BRIEF DESCRIPTION OF DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

FIG. 1 is a block diagram illustrating a schematic representation of an automatic physician assistant system in accordance with a preferred embodiment of the present disclosure.

FIG. 2 is a flow chart of an automatic physician assistant method in accordance with the present disclosure.

Various embodiments will hereinafter be described in accordance with the appended drawings, which are provided to illustrate, and not to limit the scope in any manner, wherein like designations denote similar elements, and in which:

### DETAILED DESCRIPTION

The present subject matter is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented, and the needs of a particular application may yield multiple alternate and suitable approaches to implement the functionality of any detail described herein. Therefore, any approach may extend beyond the particular implementation choices in the following embodiments described and shown.

The present application provides an AI Physician Assistant System (AI-PAS) that employs natural language processing (NLP), machine learning (ML), and data integration techniques to support physicians during patient encounters. The system captures conversation data, transcribes it, and uses NLP to identify and categorize clinical concepts. These concepts are then correlated with the patient's historical data to generate contextually relevant suggestions. The physician's responses to these suggestions are recorded and used to refine a unique profile in the doctor-specific model, which influences future suggestions and the generation of post-visit summaries. The doctor-specific model may be trained on a comprehensive dataset of anonymized patient-doctor interactions and associated clinical outcomes. The unique profile for each physician may include their specialty, historical treatment decisions, preferred medications, and any other relevant clinical preferences.

For the purpose of this description, the terms ‘physician’ and ‘doctor’ are interchangeably used.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

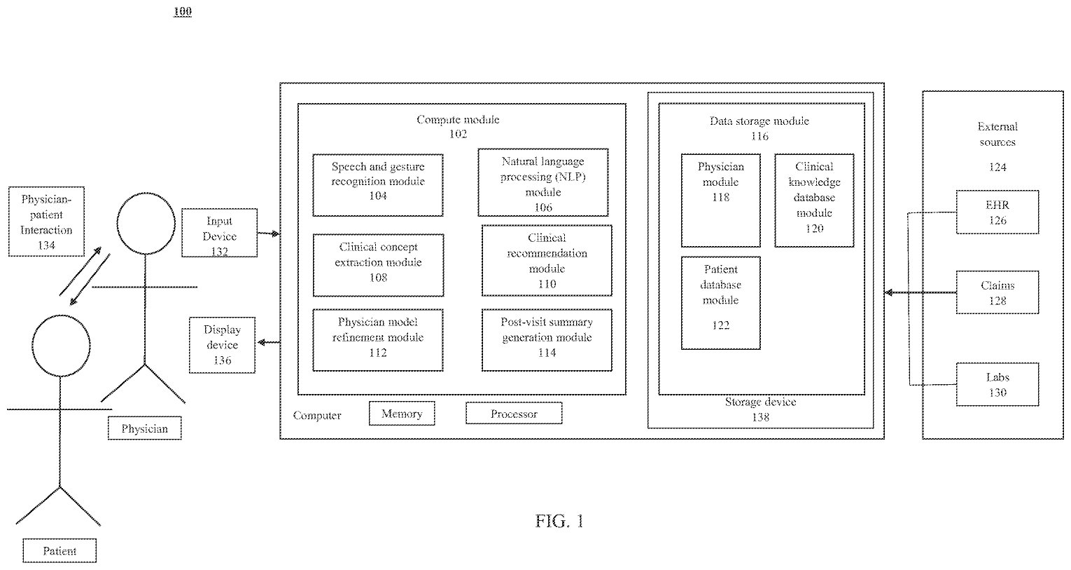

FIG. 1 is a block diagram illustrating a schematic drawing of an automatic physician assistant system 100 in accordance with the present disclosure. In a preferred embodiment, the automatic physician assistant system 100 includes a computerized device having a memory and a processor, where the automatic physician assistance system 100 is configured to provide support to physicians during patient encounters. In some embodiments, the system 100 includes a natural language processing module 106 or machine learning or data integration techniques to support physicians during patient encounters. The automatic physician assistant system 100 is configured to use an input device 132 to capture the conversation data, or the audio of the physician-patient interaction 134 between the doctor and the patient. The automatic physician assistant system 100 is also configured to capture the conversation that includes medical language and the reaction of the patient. The automatic physician assistant system 100 of the present disclosure comprises a plurality of modules. For example, and by no way limiting the scope of the present disclosure, the automatic physician assistant system 100 comprises a compute module 102 and a data storage module 116. In a preferred embodiment, the compute module 102 comprises but is not limited to a speech and gesture recognition module 104, a natural language processing (NLP) module 106, a clinical concept extraction module 108, a clinical recommendation module 110, a physician model refinement module 112 and a post-visit summary generation module 114. In yet another preferred embodiment, the data storage module 116 comprises but is not limited to a physician module 118, a clinical knowledge database module 120 and a patient database module 122.

In some embodiments, the automatic physician assistant system 100 comprises a software application that employs artificial intelligence (AI) technologies, which comprises the natural language processing (NLP) module 106 and machine learning, to assist healthcare professionals in providing medical care. The automatic physician assistant system 100 may assist with a plurality of tasks including but not limited to diagnosing diseases, interpreting medical images, managing patient data, and providing treatment recommendations. The speech and gesture recognition module 104, NLP module 106, clinical concept extraction module 108, and clinical recommendation module 110 are responsible for initial processing of transcribed text. The modules function to normalize data, filter extraneous information, and identify pivotal clinical concepts. Features may be analyzed using a suite of machine learning algorithms, which may include decision trees and neural networks, to generate contextually relevant suggestions.

The automatic physician assistant system 100 improves the efficiency and accuracy of healthcare delivery by automating routine tasks, assisting with complex decision-making, and providing access to up-to-date medical knowledge and guidelines. The automatic physician assistant system 100 also reduces healthcare costs by streamlining processes and reducing the risk of errors.

In some embodiments, the speech and gesture recognition module 104 is a component that enables the physician assistant system 100 to understand and interpret spoken commands and gestures from the user, particularly the physician. The speech and gesture recognition module 104 is configured to capture audio from patient-physician interactions and transcribe the audio into text in real-time with high accuracy. In an embodiment, the speech and gesture recognition module 104 may also be configured to interpret and understand physician's speech and gestures. In some embodiments, the speech and gesture recognition module 104 comprises computer vision to recognize and process speech and gestures, thereby allowing for more natural and intuitive interaction with the system 100.

In some embodiments, the speech and gesture recognition module 104 may be employed in interactive systems comprising smart home devices, virtual reality systems, and robotics, to enable more natural and intuitive communication between humans and machines. In some embodiments, the speech and gesture recognition module 104 comprises a combination of one or more input devices 132, such as microphones and one or more cameras capturing visible, near infrared, and infrared images to capture speech and gestures of the physician and/or the patient. In a preferred embodiment, the speech and gesture recognition module 104 uses software algorithms to process and interpret the input from said one or more microphones and one or more cameras.

In some embodiments, by incorporating the speech and gesture recognition module 104, the physician assistant system 100 can offer hands-free and voice-controlled operation, which may be especially useful in healthcare settings where clinicians need to maintain sterility or have their hands occupied with other tasks. In some embodiments, the speech and gesture recognition module 104 improves the accessibility of the system 100 for users with disabilities or limitations that make traditional input methods challenging.

In an embodiment, the natural language processing (NLP) module 106 is configured to diarize the transcribed text to attribute speech to the correct speaker. The natural language processing (NLP) module 106 enables the user to understand and process human language in a way that is similar to how humans understand language. In some embodiments, the NLP module 106 may be employed in a wide range of applications and may comprise virtual assistants, chatbots, machine translation, and text analysis.

In an embodiment, the natural language processing (NLP) module 106 is configured to identify and classify entities such as diseases, medications, and procedures mentioned in medical texts containing complex medical language or colloquial speech used in more informal or familiar conversation. Training models on large datasets of medical texts and colloquial speech improves performance in understanding and generating texts. In some embodiments, the specialized knowledge bases and ontologies in the medical domain enhance the understanding and processing of medical language. Medical ontologies and codes may relate to the International Classification of Diseases and related codes and the Current Procedural Terminology (CPT®) system.

The natural language processing (NLP) module 106 enables the physician assistant system 100 to understand and interpret human language. In an embodiment, the natural language processing (NLP) module 106 comprises NLP algorithms and models to analyze text or speech input from the user, allowing the system 100 to extract meaning, identify key information, and generate appropriate responses. The appropriate responses may be presented to the physician using a display device 136.

In some embodiments, the NLP module 106 may be configured for various purposes, and comprises understanding and processing clinical notes, patient histories, and other medical documents to extract relevant information. In an embodiment, interpreting spoken commands or queries from the user to perform tasks comprises retrieving patient information, providing treatment recommendations, or scheduling appointments, generating written reports or summaries based on clinical data or interactions with the patient, and supporting clinical decision-making by providing relevant information, guidelines, or alerts based on the context of the conversation.

The clinical concept extraction module 108 is configured to identify and extract clinical concepts from the conversation comprising but not limited to clinical terms, patient symptoms, diagnostic information, and treatment options discussed or likely to be discussed during the patient-doctor interaction. The clinical concept extraction module 108 focuses on identifying and extracting clinical concepts from text, such as electronic health records (EHRs) 126 or medical literature. These units are designed to recognize specific medical terms, conditions, treatments, and other relevant information that is crucial for clinical decision-making and research.

In some embodiments, the clinical concept extraction module 108 employs natural language processing (NLP) that involves identifying and extracting relevant clinical information from text, comprising medical records, clinical notes, or research articles. In some embodiments, the clinical concept extraction module 108 is important for converting unstructured clinical text into structured data that can be used for various applications in healthcare, such as clinical decision support, data mining, and research.

In some embodiments, the clinical concept extraction module 108 is configured to (i) remove noise and irrelevant information from the text, format characters and punctuation, (ii) break the text into individual words or tokens, (iii) identify and categorize specific entities in the text, such as medical terms, symptoms, diseases, treatments, and drug names, (iv) identify relationships between entities, such as the relationship between a symptom and a disease, (v) analyze the meaning of the text to infer additional information, such as the severity of a symptom or the context of a diagnosis.

The clinical recommendation module 110 is configured to integrate real-time data with historical patient records and analyze the combined data set to generate evidence-based suggestions to the physician through a user-friendly interface via a display device. The clinical recommendation module 110 is also configured to provide recommendations or suggestions to healthcare providers based on clinical guidelines, best practices, and patient-specific data. In a preferred embodiment, the module 100 leverages artificial intelligence (AI) and machine learning (ML) techniques to analyze patient data, such as medical records, diagnostic tests, and treatment histories, to generate personalized recommendations for diagnosis, treatment, and follow-up care.

In some embodiments, the clinical recommendation module 110 is configured to provide evidence-based recommendations to healthcare providers to assist them in making clinical decisions. The evidence-based suggestions may be presented to physicians on a display device 136. In some embodiments, the clinical recommendation module 110 uses algorithms and guidelines based on clinical knowledge and research to suggest appropriate diagnostic tests, treatments, or interventions for specific patient conditions. In some embodiments, the clinical recommendation module 110 may comprise a plurality of healthcare settings that include but are not limited to hospitals, clinics, and telemedicine platforms to support healthcare providers in delivering high-quality care. In some embodiments, the clinical recommendation module 110 may reduce errors, improve adherence to best practices, and enhance patient outcomes by providing timely and relevant recommendations based on the latest clinical evidence. The system provides real-time guidance to healthcare providers during patient encounters, suggests appropriate diagnostic tests or treatment options based on the patient's condition and medical history. In some embodiments, this module is configured to assist healthcare providers in developing individualized treatment plans for patients, and to consider factors such as disease severity, comorbidities, and patient preferences.

The physician model refinement module 112 focuses on improving the performance and accuracy of AI models used in clinical decision-making. The physician model refinement module 112 is configured to refine and optimize AI models based on feedback from healthcare providers, new research findings, evolving clinical guidelines, and incorporating feedback from healthcare providers to correct errors and improve the performance of AI models. In some embodiments, the feedback may include but is not limited to annotations, corrections, and explanations provided by physicians during model validation. In some embodiments, the physician model refinement module 112 is configured to update AI models with new data and insights to ensure they remain up-to-date with the latest clinical knowledge and practices.

In some embodiments, the physician model refinement module 112 uses feedback from healthcare providers to improve the accuracy and effectiveness of AI models used in clinical decision-making. In some embodiments, the physician model refinement module 112 is configured to continuously learn and adapt based on real-world data and expert input to refine the AI models to better align with clinical practice, including the documentation style of the physician, and improve patient outcomes. In some embodiments, the physician model refinement module 112 is configured to gather feedback from healthcare providers on the performance of the AI models, including any discrepancies between the model's recommendations and clinical practice. In some embodiments, the physician model refinement module 112 uses feedback to retrain the AI models using updated data and algorithms to improve the model's accuracy and relevance to clinical practice. In some embodiments, the physician model refinement module 112 is configured to assess the performance of the refined AI models using metrics such as sensitivity, specificity, and accuracy, as well as to evaluate their impact on clinical outcomes. In some embodiments, the refined AI models are deployed back into the healthcare system for use by healthcare providers along with mechanisms for monitoring their performance and collecting further feedback.

The physician model refinement module 112 is configured to (a) create a unique profile for each physician, (b) track every interaction between the physician and the AI system on the computerized device, (c) analyze the feedback from the physician to understand their decision-making patterns, (d) update the physician's profile based on the analyzed feedback with the help of an algorithm, (e) continuously learn from each physician interaction, allowing it to refine the unique profile in the doctor-specific model over, time and (f) forecast future physician decisions based on past behavior with the help of a predictive analytics, thereby improving the relevance of suggestions over time. The feedback analysis may include the clinical context in which suggestions were accepted or rejected. Continuous learning may include a model update mechanism and algorithm that includes reinforcement learning techniques where the model is rewarded for suggestions that align with the physician's actions. The model may be maintained and updated based on physician feedback locally on a single computerized device, which allows the computerized device to operate in high security and safety environments.

The post-visit summary generation module 114 is configured to compile a comprehensive visit summary based on the conversation and the physician's actions. The post-visit summary generation module 114 is also configured to automatically generate summaries of patient visits after they have occurred. These summaries typically include but are not limited to key information discussed during the visit, such as diagnoses, treatments, medications prescribed, follow-up instructions, and any other relevant information. The post-visit summary generation module 114 is configured to extract relevant information from electronic health records (EHRs) 126, including but not limited to clinical notes, test results, and medication lists. The post-visit summary generation module 114 is configured to analyze and summarize the extracted information using NLP techniques to generate a coherent and concise summary of the visit. In a preferred embodiment, the post-visit summary generation module 114 comprises predefined templates to structure the comprehensive visit summary and ensure that all relevant information is included, thereby allowing healthcare providers to customize the comprehensive visit summary based on their preferences and the specific needs of the patient. The post-visit summary generation module 114 may be configured to use the learned documentation style as an input into the format of the comprehensive visit summary.

The data storage module 116 is configured to store and manage the data used by the system 100. In a preferred embodiment, the data storage module 116 comprises a database or data repository where various types of data relevant to healthcare including patient records, medical images, lab results, and treatment plans, are stored in a structured format. The data storage module may be located on a storage device 138.

The data storage module 116 ensures the security, integrity, and accessibility of the data. The data storage module 116 comprises features for data encryption, access control, and data backup to protect against data loss and unauthorized access. In an embodiment, the data storage module 116 comprises mechanisms for data retrieval and querying, thereby allowing healthcare providers to access and retrieve relevant information quickly and efficiently.

In some embodiments, in addition to storing and managing clinical data, the data storage module 116 may also store administrative and operational data, such as billing information, scheduling data, and inventory management information, to support the overall functioning of the healthcare system.

The data storage module 116 is configured to store data of the physician and patient. In an embodiment, the data storage module 116 comprises the physician module 118. The physician module 118 in the context of healthcare and artificial intelligence (AI), typically refers to a computational model or algorithm that is trained to perform tasks that are traditionally carried out by physicians. The physician module 118 may range from simple decision trees to complex deep learning algorithms and is configured to assist healthcare providers in various aspects of clinical practice, diagnosis, treatment planning, and patient management. In a preferred embodiment, the physician module 118 is developed using machine learning techniques and is trained on large datasets of medical records, imaging studies, and other healthcare data. The physician model 118 may be configured to analyze complex patterns in the data to identify potential diagnoses, predict patient outcomes, and recommend personalized treatment plans. The physician module 118 has the potential to improve the efficiency and accuracy of healthcare delivery by providing healthcare providers with timely and evidence-based recommendations. In some embodiments, the physician module 118 raises important ethical and regulatory considerations, thereby ensuring patient privacy and safety, and maintaining the human-centric nature of healthcare.

The patient database module 122 is a structured collection of data related to patients' health and medical history. In a preferred embodiment, the patient database module 122 comprises information such as patient demographics, medical conditions, medications, allergies, test results, and treatment plans. Patient databases are commonly used in healthcare settings to store and manage patient information, allowing healthcare providers to access and update patient records as needed.

The patient databases module 122 is an important component of electronic health record (EHR) 126 to store and manage patient health information electronically. The EHR 126 enables healthcare providers to access patient records from different locations, share information with other providers, and track patient progress over time.

In some embodiments, in addition to the EHR 126, the patient databases module 122 may also be used in research settings to collect and analyze data for clinical studies and epidemiological research. These databases can help researchers identify trends, evaluate treatment outcomes, and improve patient care.

The clinical knowledge database module 120 is a repository of structured and unstructured information related to clinical medicine and healthcare. The clinical knowledge database module 120 comprises a wide range of data, including medical terminology, disease classifications, diagnostic criteria, treatment guidelines, drug information, and clinical pathways. The clinical knowledge database module 120 is used by healthcare professionals to access up-to-date information, make informed decisions about patient care, and stay informed about the latest developments in medicine. In an embodiment, the clinical knowledge database module 120 may integrate into electronic health record (EHR) 126, clinical decision support systems, and other healthcare IT applications to provide clinicians with relevant information at the point of care. In some embodiments, the clinical knowledge database 120 is continuously updated to reflect the latest advancements in medical research and clinical practice. The clinical knowledge database 120 is an important tool for improving the quality, efficiency, and safety of patient care by ensuring that healthcare providers have access to accurate and current information.

In an embodiment, the external sources 124 comprise but are not limited to data from the electronic health records (EHR) 126, claims 128, and lab records 130 which are accessible by the system 100 using a communication device, a network, or another communication connection. In an embodiment, the lab record data 130 comprises but is not limited to blood tests, imaging results, and other diagnostic tests that provide valuable information to the physician assistant system. The system helps healthcare providers make more accurate diagnoses and develop appropriate treatment plans using the analyzed lab record data 130. In an embodiment, the system uses the labs data 130 and the claims data 128 to identify appropriate treatments based on the patient's medical history, insurance coverage, and other relevant factors. In an embodiment, the labs data 130 and claims data 128 may be used by the system to monitor patients' progress over time and track the effectiveness of treatments. In an embodiment, the system is configured to alert healthcare providers to any abnormalities or changes in the data that may require further evaluation or intervention. In another embodiment, the system is configured to analyze the labs data 130 and the claims data 128 across a population of patients and identifies trends, risk factors, and areas for improvement in healthcare delivery. The information is used to develop targeted interventions and improve overall population health.

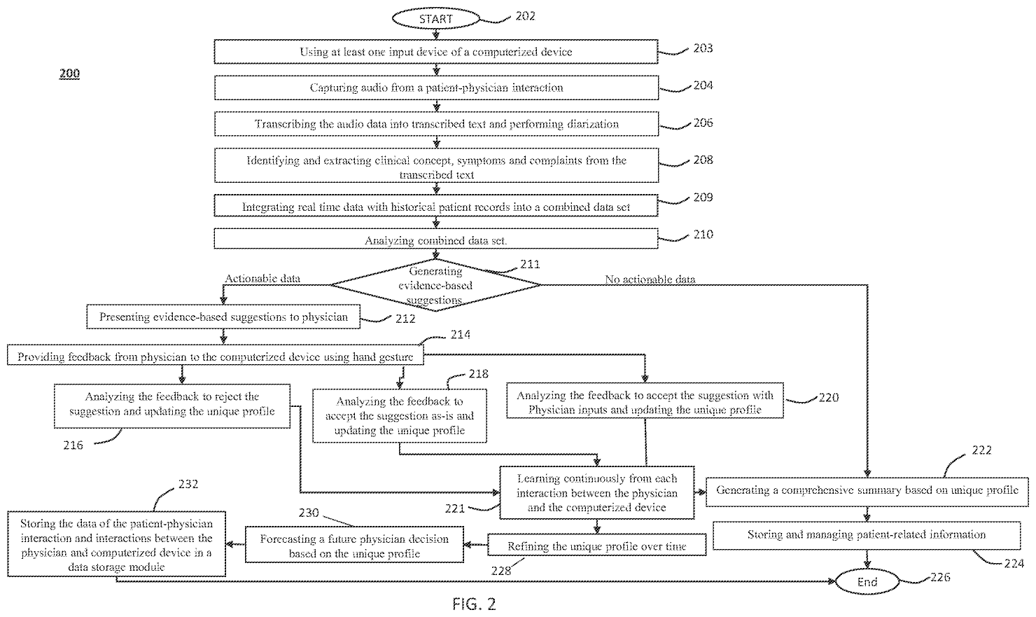

FIG. 2 illustrates a flowchart of an automatic physician assistant method 200 according to an embodiment of the present subject matter. At step 202, the system starts encountering a patient. At step 203 the system uses at least one input device of a computerized device. The computerized device has at least one non-transitory memory and at least one processor capable of executing instructions in the memory. The at least one input device is in communication with the memory. The input device is configured to capture at least one of audio data or image data. At step 204, the system starts listening to the conversation between the doctor and the patient using AI technique, capturing audio data from the physician-patient interaction. At step 206, the system transcribes the audio data into transcribed text and performs diarization. At step 208, the system identifies and extracts clinical concepts, symptoms, and complaints from the transcript from the transcribed text. At step 209, real time data is integrated with historical patient records into a combined data set. At step, 210, based on the extracted data, the AI scribe searches the clinical knowledge base and patient history and analyzes the combined data set to identify the next step. At step 211, the system checks if there is any actionable data to use in generating evidence-based suggestions. If yes, then the evidence-based suggestions based on the actionable data are presented to the physician at step 212. Thereafter, the physician provides feedback to the computerized device by using hand gestures at step 214 to accept or reject the suggestion. The feedback is analyzed. If the suggestion is rejected, the method 200 proceeds from step 212 to step 216 where the unique profile is updated without adding the suggestions. If, however, the suggestion is accepted by the physician as is, the method 200 proceeds from step 212 to step 218 where the suggestion is accepted as-is and the unique profile is updated accordingly. In some embodiments, the physician may accept the suggestions but with some inputs for editing the suggestion. The method in such embodiments proceeds from step 212 to step 220, where the edited suggestion is utilized for updating the unique profile. At step 221 continuous learning occurs from each interaction between the physician and the computerized device. Thereafter, a comprehensive visit summary is generated based on the unique profile at step 222. Looping back to step 211, if there is no actionable data, the method 200 proceeds directly to step 222 for generating the comprehensive visit summary. Thereafter, at step 224, patient-related information is stored and managed, for example by sending a summary note to EHR for updating the records. The unique profile is refined over time at step 228. At step 230, a future physician decision may be forecasted based on the unique profile. At step 232, the data of the patient-physician interaction and interactions between the physician and computerized device is stored in a data storage module. The method 200 ends at step 226.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

It is noted that various connections are set forth between elements in the description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

Various embodiments of the disclosure have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprise” and “comprising” should be interpreted as referring to elements, components, or steps, in a non-exclusive manner, indicating that the referenced elements, components, or steps may be present, or utilized, or combined with other elements, components, or steps that are not expressly referenced.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices, or arrangements of devices that are capable of implementing the steps that constitute the method of the disclosure.

The computer system comprises a computer, an input device, a display device, and the Internet. The computer further comprises a microprocessor. The microprocessor is connected to a communication bus. The computer also includes memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as reception of data from other sources. The communication unit may include a modem, an Ethernet card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source, or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C”, “C #”, “C+”, “C++”, “Embedded C”, “Visual C++,” Java”, “Python” and “Visual Basic”. Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “iOS”, “Mac” “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage device, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The claims can encompass embodiments for hardware, software, or a combination thereof.

Although a few implementations have been described in detail above, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

We claim:

1. A computer-implemented system for automatically assisting physicians during patient encounters, the system comprising:
a computerized device having at least one non-transitory memory and at least one processor capable of executing instructions stored in the memory;
at least one input device in communication with the memory, wherein the input device is configured to capture at least one of audio data or image data;
a compute module stored on the memory and configured to process, interpret and analyze data to provide accurate and timely assistance to physicians, the compute module comprising:
a speech and gesture recognition module configured to received audio data from a patient-physician interaction captured by the input device, and transcribe the audio data into transcribed text in real time;
a natural language processing module configured to diarize the transcribed text to attribute speech to a correct speaker;
a clinical concept extraction module configured to identify and extract clinical concepts from the transcribed text; and
a clinical recommendation module configured to integrate real-time data with historical patient records into a combined data set, and analyze the combined data set to generate evidence-based suggestions to the physician, wherein the evidence-based suggestions are provided to the physician through a display device.

2. The system of claim 1, wherein the speech and gesture recognition module is configured to transcribe audio data which includes one or more accents, dialects, or medical terminologies, and configured to recognize hand gestures and convert said hand gestures to a command for accepting and rejecting the evidence-based suggestions.

3. The system of claim 1, wherein the audio data comprises at least one of: clinical terms, patient symptoms, diagnostic information, or treatment options discussed during the patient-physician interaction.

4. The system of claim 1, wherein the compute module further comprises:
a physician model refinement module configured to train and validate using historical data of the physician, the physician model refinement module configured to:
create a unique profile for each physician;
track interactions between the physician and the computerized device;
analyze feedback from the physician to understand decision-making patterns;
update the unique profile of the physician based on the analyzed feedback;
learn continuously from each interaction between the physician and the computerized device, and refine the physician model over time; and
forecast a future physician decision based on the unique profile using predictive analytics to improve a relevance of the evidence-based suggestions over time.

5. The system of claim 1, wherein the compute module further comprises a post-visit summary generation module configured to compile a comprehensive visit summary of the patient-physician interaction based on the audio data and the image data of the physician.

6. The system of claim 1, further comprising:
a data storage module configured to store data of the patient-physician interaction and interactions between the physician and the computerized device, the data storage module comprising:
a physician module comprising at least one of physician feedback, notes, medications or cases;
a clinical knowledge database module comprising a structured collection of information related to clinical medicine and healthcare, and further comprising at least one of: medical terminology, disease classifications, diagnostic criteria, treatment guidelines, drug information, or clinical pathways; and
a patient database module configured to store and manage patient-related information, the patient-related information comprising at least one of: patient demographics, medical history, diagnoses, treatments, medications, or lab results.

7. The system of claim 1, further comprises an external source of electronic health records (EHRs) enabling the clinical recommendation module to incorporate at least a medical history of the patient into the evidence-based suggestions.

8. The system of claim 1, wherein the natural language processing module is configured to process complex medical language and colloquial speech.

9. The system of claim 1, wherein the clinical concept extraction module is configured to map extracted clinical concepts to standardized medical ontologies and codes.

10. The system of claim 5, wherein the post-visit summary generation module is configured to format the comprehensive visit summary according to a learned documentation style of the physician, to allow for physician review and editing before finalizing and sending the comprehensive visit summary to the EHRs.

11. A computer-implemented method for automatically assisting physicians during patient encounters, the method comprising:
using at least one input device of a computerized device, the computerized device having at least one non-transitory memory and at least one processor capable of executing instructions stored in the memory, and the at least one input device being in communication with the memory and being configured to capture at least one of audio data or image data;
capturing audio data from a patient-physician interaction and transcribing the audio data into transcribed text in real-time;
diarizing the transcribed text to attribute speech to a correct speaker using a natural language processing module;
identifying and extracting clinical concepts from the transcribed text, the transcribed text comprising at least one of: clinical terms, patient symptoms, diagnostic information, and treatment options discussed during the patient-physician interaction; and
integrating real-time data with historical patient records to form a combined data set, and analyzing the combined data set to generate evidence-based suggestions, whereby the evidence-based suggestions are provided to the physician through a display device.

12. The method of claim 11, further comprising the steps of:
providing feedback from the physician to the computerized device;
creating a unique profile for each physician and tracking interactions between the physician and the computerized device;
analyzing the feedback from the physician to understand decision-making patterns of the physician to at least one of: reject, accept, or accept the evidence-based suggestions with physician inputs;
updating the unique profile of the physician based on analyzed feedback;
learning continuously from each interaction between the physician and the computerized device, thereby refining the unique profile over time;
forecasting a future physician decision based on the unique profile, thereby improving a relevance of the evidence-based suggestions over the time;
storing data of the patient-physician interaction and interactions between the physician and the computerized device in a data storage module; and
generating a comprehensive visit summary based on the unique profile.

13. The method of claim 11, wherein the data storage module comprises at least one of: physician feedback, notes, medications, or cases.

14. The method of claim 11, wherein the data storage module further comprises a clinical knowledge database module comprising at least one of: medical terminology, disease classifications, diagnostic criteria, treatment guidelines, drug information, or clinical pathways.

15. The method of claim 11, further comprising the step of storing and managing patient-related information by a patient database module, wherein the patient-related information comprises at least one of: patient demographics, medical history, diagnoses, treatments, medications, or lab results.
